(19) United States
(12) Patent Application Publication
(10) Pub. No.: US 2012/0165839 A1
(43) Pub. Date: Jun. 28, 2012

(54) Medical Needle Holder

(76) Inventors: Nicolas Haseke, Muenchen (DE); Sven Schneider, Tuttlingen (DE); Uwe Bacher, Tuttlingen (DE)

(21) Appl. No.: 13/336,646

(22) Filed: Dec. 23, 2011

(30) Foreign Application Priority Data
Dec. 23, 2010 (DE) 10 2010 055 806.0

(51) Int. Cl. A61B 17/04

(52) U.S. Cl. 606/147

(57) ABSTRACT

A medical needle holder with a shaft, a tool that is mounted on the distal end of the shaft and two jaw members, such that one jaw member of the tool is configured to pivot with respect to the other jaw member of the tool, as well as with a handle that is mounted on the proximal end of the shaft and at least two gripping members, such that the pivotable jaw member of the tool can be actuated by a movably configured gripping member of the handle, such that at least one portion of at least one jaw member of the tool is configured to slide with respect to the other jaw member of the tool in the longitudinal direction of the shaft. The slidable jaw member or the slidable portion of the jaw member is pre-tensed in a starting position by a spring element.

### CROSS-REFERENCE TO RELATED APPLICATIONS

The present application claims priority of German patent application No. 10 2010 055 806.0 filed on Dec. 23, 2010.

### FIELD OF THE INVENTION

The invention relates to a medical needle holder with a shaft, with a tool positioned on the distal end of the shaft consisting of two jaw members such that one jaw member of the tool is configured to pivot with respect to the other jaw member, and with a handle positioned on the proximal end of the shaft comprising at least two gripping members, such that the pivotable jaw member of the tool can be actuated by a movably configured gripping member of the handle, and at least one portion of at least one jaw member of the tool is configured so that it can slide with respect to the other jaw member of the tool in the longitudinal direction of the shaft.

### BACKGROUND OF THE INVENTION

Medical needle holders are known in various embodiments. Medical needles, which as a rule are curved in a sickle shape, are gripped by the tool and held clamped between the jaw members of the tool. To align the needle, held in such a manner, in the correct position for suturing a surgical site, the operator must either manually align the needle or else move it to the required position between the jaw members of the tool by means of an additional gripping forceps.

A generic medical needle holder is known, for example, from patent DE 91 09 097 U1.

### SUMMARY OF THE INVENTION

Consequently, it is the object of the invention to provide a medical needle holder that allows easy positioning of the needle in the tool.

This object is fulfilled, according to the invention, in that the slidable jaw member or the slidable portion of the jaw member is pre-tensed in a starting position by a spring element.

Owing to the configuration of the jaw members of the tool, according to which at least one portion of at least one jaw member of the tool is configured to slide with respect to the other jaw member of the tool in the longitudinal direction of the shaft, it is possible for the first time to align the position of the surgical needle held between the jaw members without recourse either to an additional tool or to manual gripping. After release of the surgical needle, to allow the slidable jaw member the same freedom of movement for renewed gripping of the needle, the slidable jaw member or slidable portion of the jaw member is, according to the invention, pre-tensed in a starting position by a spring element.

According to a practical embodiment of the invention, it is proposed that at least one portion of the non-pivotable jaw member of the tool should be configured to slide in the longitudinal direction of the shaft. It is particularly simple to achieve this longitudinal slidability in constructing the non-pivotable jaw member, because this jaw member is not required to execute any further movements, contrary to the pivotable jaw member.

The jaw member or portion of a jaw member is slid in the longitudinal direction of the shaft, according to the invention, by a power drive mounted on the handle. With a preferred embodiment of the invention, it is proposed that the power drive should be configured as an adjusting wheel.

To configure the axially slidable jaw member, it is further proposed with the invention that the slidable jaw member or the slidable portion of the jaw member should constitute the distal end of a slide that is movable in the longitudinal direction of the shaft and is mounted in a guiding position on the shaft. The guided slide ensures that the jaw member can be slid precisely and without risk of tipping.

To power the slide in the longitudinal direction of the shaft, the rotary movement generated by the power drive configured as an adjusting wheel is converted into a purely axial longitudinal motion of the slide. This transmission can be achieved, for example, by a purely friction-locked connection or else by a toothed interlocking of the adjusting wheel and slide.

To minimize the wear of the gripping surface of the slidable jaw member in the area of contact between the surgical needle and the slidable jaw member, at least the portion of the gripping surface of the slide that serves to receive a surgical needle that is to be held should consist of a hard metal.

In addition, it is proposed with the invention that to power the portion of a jaw member that is to be slid in the longitudinal direction of the shaft, a power drive that is preferably configured as a setting screw should be positioned on the distal end of this jaw member.

To ensure, on the one hand, that the surgical needle can be mounted to be easily rotatable between the two jaw members of the tool and, on the other hand, that the surgical needle can be easily and securely fastened between the jaw members of the tool, it is proposed according to a first embodiment of the invention that a replaceable plastic track should be positioned running in the longitudinal direction of the jaw member on the gripping surface of at least one jaw member that serves to receive a surgical needle that is to be held. The track, on the one hand, minimizes the contact surface of the needle with the jaw members and, on the other hand, because of the malleability of the plastic material, allows the needle to be held in place securely when the two jaw members are pressed together by the handle.

With a second inventive embodiment it is proposed that a replaceable plastic covering should be placeable on the gripping surface of at least one jaw member that serves to receive a surgical needle that is to be held. This variant can be assembled and replaced especially rapidly and easily, because the plastic covering is applied from outside onto the at least one jaw member.

In addition, with a third alternative embodiment, it is proposed that at least the gripping surface of at least one jaw member that serves to receive a surgical needle that is to be held should consist of a replaceable plastic material. With this embodiment, at least the gripping surface, if not the entire jaw member as well, consists of a plastic material.

It is finally proposed with the invention that the gripping members of the handle should be capable of being fixed in their respective position to one another in order, on the one hand, to ensure a constantly secure grasp of the surgical needle in the tool and, on the other hand, to relieve the operator, so that he or she is not obliged to actuate the gripping members continuously.

Additional characteristics and advantages of the invention can be seen from the appended drawings, in which various embodiments of an inventive medical needle holder are depicted only by way of example, without restricting the invention to these embodiments.

### BRIEF DESCRIPTION OF THE DRAWINGS

FIG. 1 shows a side view of a first embodiment of an inventive medical needle holder with the jaw members in a first working position.

FIG. 2 shows a depiction according to FIG. 1 but showing the jaw members in a second working position.

FIG. 3 shows a perspective view of the distal end of a second embodiment of an inventive medical needle holder in the opened position.

FIG. 4 shows an enlarged longitudinal section along the line IV-IV in FIG. 3.

FIG. 5 shows an enlarged depiction of detail V of FIG. 1, showing a first embodiment of the jaw members.

FIG. 6 shows a section along the line VI-VI in FIG. 5.

FIG. 7 shows an enlarged depiction of detail VII of FIG. 1, showing a second embodiment of the jaw members.

FIG. 8 shows a section along the line VIII-VIII in FIG. 7.

FIG. 9 shows an enlarged depiction of detail IX of FIG. 1, showing a third embodiment of the jaw members.

### DETAILED DESCRIPTION OF THE INVENTION

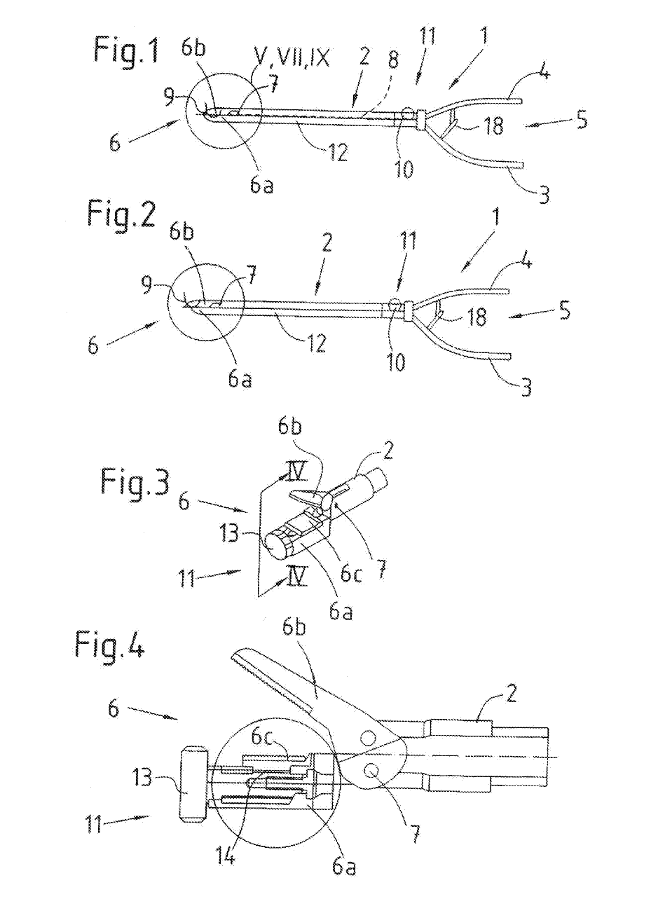

FIGS. 1 and 2 show the side views of a medical needle holder 1.

The illustrated medical needle holder 1 consists essentially of a hollow shaft 2 on whose proximal end a handle 5 is positioned comprising two gripping members 3 and 4, such that in the illustrated embodiment the gripping member 3 is mounted on the handle 5 so that it can be pivoted with respect to the other gripping member 4.

Positioned on the distal end of the shaft 2 is a tool 6, which in the illustrated embodiment consists of a jaw member 6a rigidly connected with the shaft 2 and a jaw member 6b that can pivot around a pivot axis 7. To open and close the jaw members 6a and 6b by means of the actuation of the pivotable gripping member 3 of the handle 5, the pivotable gripping member 3 and the pivotable jaw member 6b are in operative connection with one another via a push-pull rod 8 mounted in the hollow shaft 2.

As can be seen in particular from FIGS. 1 through 3, the jaw members 6a and 6b of the tool 6 are not only configured in such a way that the jaw member 6b can be pivoted with respect to the jaw member 6a, but in addition the jaw member 6a or a portion 6c of the jaw member 6a is configured so that it can slide with respect to the jaw member 6b in the longitudinal direction of the shaft 2.

Alternatively to the illustrated configuration of the jaw members 6a and 6b of the tool 6, it is also possible of course for the upper jaw member 6b to be configured rigidly, that is, not to pivot, while the lower jaw member 6a is of pivotable configuration. Likewise, the jaw member 6b or a portion of the jaw member 6b can be configured to slide with respect to the jaw member 6a in the longitudinal direction of the shaft 2. A relative slidability of the two jaw members 6a and 6b with respect to one another is also a possible construction variant.

As a result of this configuration of the jaw members 6a and 6b of the tool 6, whereby at least a portion 6c of at least one jaw member 6a or 6b of the tool 6 is configured to slide in relation to the other jaw member 6b or 6a of the tool 6 in the longitudinal direction of the shaft 2, it is possible to align the position of a surgical needle 9 held between the jaw members 6a and 6b without resorting to an additional tool or to manual gripping, because the sliding of the jaw members 6a and 6b in relation to one another causes a rotation of the needle 9 around its longitudinal axis.

In the medical needle holder 1 illustrated in FIGS. 1 and 2, the sliding of the rigid jaw member 6a, that is, the one that is not pivotable, in the longitudinal direction of the shaft 2 is effected by the fact that the entire lower part of the shaft 2, whose distal end constitutes the jaw member 6a, is configured to slide in relation to the upper part of the shaft 2 by means of a power drive 11 configured as an adjusting wheel 10. For this purpose, the slidable lower part of the shaft 2 constitutes a slide 12, which is mounted in a guiding position on the upper part of the shaft 2. The guided slide 12 ensures a precise, tip-proof axial sliding of the jaw member 6a.

To drive the slide 12 in the longitudinal direction of the shaft 2, the rotary motion generated by the power drive 11 configured as an adjusting wheel 10 is converted into a purely axial longitudinal motion of the slide 12. This transmission can be effected, for example, by a purely friction-locked connection or else by a toothed connection of the adjusting wheel 10 and slide 12.

In the alternative embodiment, illustrated in FIGS. 3 and 4, for configuring the relative slidability of the jaw members 6a and 6b of the tool 6, the entire jaw member 6a is not configured to slide in the longitudinal direction of the shaft 2, but instead only the portion 6c of the jaw member 6a that serves for the clamping reception of the needle 9.

In this embodiment, the power drive 11 of the slidable portion 6c of the jaw member 6a is configured as a setting screw 13, which is positioned on the distal end of the jaw member 6a and is in operative connection with the slidable portion 6c via a toothed connection 14.

To minimize wear on the gripping surface of the slidable jaw member 6a or of the slidable portion 6c in the area of contact with the surgical needle 9, at least the portion of the gripping surface of at least one jaw member 6a, 6b that serves to receive a surgical needle 9 that is to be held consists of a hard metal.

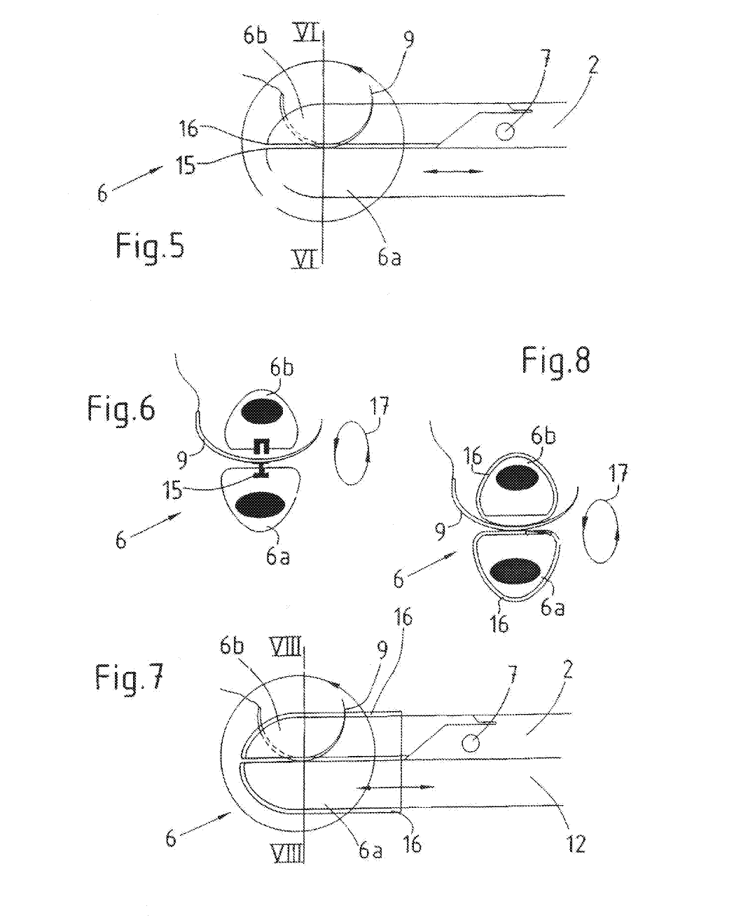

FIGS. 5 through 9 illustrate three different embodiments for configuring the jaw members 6a and 6b as they come into use in configuring the completely slidable jaw member 6a in accordance with FIGS. 1 and 2.

To ensure, on the one hand, that the surgical needle 9 can be mounted so as to rotate easily between the two jaw members 6a and 6b of the tool 6 and, on the other hand, that the surgical needle 9 can be fixed easily and securely between the jaw members 6a and 6b of the tool 6, in these embodiments a synthetic material is mounted on the gripping surface of at least one jaw member 6a or 6b that serves to receive a surgical needle that is to be held. Because of the malleability of the synthetic material, secure fastening of the needle 9 is ensured when both jaw members 6a and 6b are pressed together by a handle 5.

In the first embodiment illustrated in FIGS. 5 and 6, replaceable tracks 15 of a synthetic material, extending in the longitudinal direction of the jaw members 6a and 6b, are mounted on both gripping surfaces of the jaw members 6a and 6b that serve to receive the surgical needle 9 that is to be held. These tracks 15 minimize the contact surface of the needle 9 with the jaw members 6a and 6b and thus facilitate the rotation of the needle 9 during the sliding of the jaw member 6a.

According to the second embodiment illustrated in FIGS. 7 and 8, replaceable coverings 16 of a synthetic material can be placed on the gripping surfaces of the jaw members 6a and 6b that serve to receive the surgical needle 9 that is to be held. This variant can be installed and exchanged especially quickly and easily because the synthetic coverings 16 are placed on the jaw members 6a and 6b from outside.

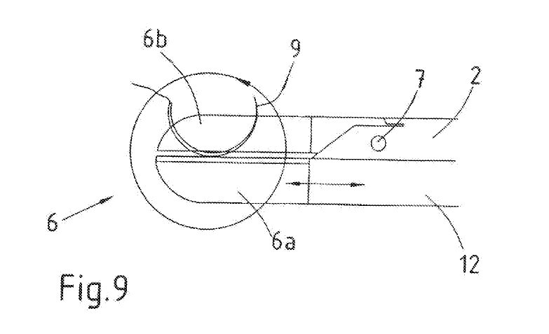

In the third embodiment illustrated in FIG. 9, the entire jaw members 6a and 6b, or at least the gripping surfaces of the jaw members 6a and 6b that serve to receive the surgical needle 9 that is to be held, consist of a replaceable synthetic material.

The medical needle holder 1 described above and illustrated in FIGS. 1 through 9 is operated as follows.

Starting from the opened position of the tool 6 shown in FIG. 3, the needle holder 1 is placed by the user in such a way that the surgical needle 9 comes to rest between the jaw members 6a and 6b of the tool 6. Then the user presses the gripping members 3 and 4 of the handle 5 together so that the pivotable jaw member 6b is closed and the needle is held clamped between the two jaw members 6a and 6b.

In order to suture a surgical site, the needle 9, which is curved in sickle form as shown in FIGS. 6 and 8, must be aligned in the needle holder 1 in such a way that the operator can insert the needle 9 at the right instant in the tissue that is to be sutured.

The needle 9 that is held between the jaw members 6a and 6b is aligned in the described needle holder 1 by sliding either the entire jaw member 6a or just a portion 6c of the jaw member 6a in relation to the other jaw member 6b in the longitudinal direction of the shaft 2, causing a rotation of the needle 9 around its longitudinal axis as is shown by the arrow 17 in FIGS. 6 and 8.

On the one hand, to ensure a constantly secure grip of the surgical needle 9 in the tool 6 and, on the other hand, to relieve the operator so that he or she is not obliged to actuate the gripping members 3 and 4 of the handle 5 continuously, the gripping members 3 and 4 of the handle 5 can be fixed in their respective position with respect to one another by a blocking mechanism 18, as can be seen from FIGS. 1 and 2.

It is possible by means of this blocking mechanism 18 to secure the needle between the jaw members 6a and 6b in a first step only in such a way that the needle can still be rotated for alignment and so that the needle 9 is clamped definitively in the proper position only thereafter.

After releasing the surgical needle 9, in order to allow the slidable jaw member 6a or the slidable portion 6c of the jaw member 6a to resume the same freedom of movement for renewed gripping of the needle 9, the slidable jaw member 6a or the slidable portion 6c of the jaw member 6a is pre-tensed in a starting position via a spring element, which is not illustrated.

A medical needle holder 1 of this configuration is characterized in that it makes it possible to position the needle 9 easily in the tool 6 without recourse to an additional instrument.

1. A medical needle holder with a shaft, with a tool that is positioned on the distal end of the shaft and consists of two jaw members, such that one jaw member of the tool is configured to pivot with respect to the other jaw member of the tool, as well as with a handle that is positioned on the proximal end of the shaft and comprises at least two gripping members, such that the pivotable jaw member of the tool can be actuated by a movably configured gripping member of the handle, such that at least one portion of at least one jaw member of the tool is configured so that it can slide with respect to the other jaw member of the tool in the longitudinal direction of the shaft,
wherein
the slidable jaw member or the slidable portion of the jaw member is pre-tensed in a starting position by a spring element.

2. The medical instrument according to claim 1, wherein at least one portion of the non-pivotable jaw member of the tool is configured to slide in the longitudinal direction of the shaft.

3. The medical instrument according to claim 1, wherein the sliding motion in the longitudinal direction of the shaft occurs by means of a power drive positioned on the handle.

4. The medical instrument according to claim 3, wherein the power drive is configured as an adjusting wheel.

5. The medical instrument according to claim 2, wherein the slidable jaw member or the slidable portion of the jaw member constitutes the distal end of a slide that can slide in the longitudinal direction of the shaft and is mounted in a guiding position on the shaft.

6. The medical instrument according to claim 4, wherein the rotary motion of the adjusting wheel can be converted into a longitudinal motion of the slide.

7. The medical instrument according to claim 5, wherein at least the portion of the gripping surface of the slide that serves to receive a surgical needle that is to be held consists of a hard metal.

8. The medical instrument according to claim 1, wherein the sliding motion of the portion of the jaw member in the longitudinal direction of the shaft occurs by means of a power drive positioned on the distal end of the jaw member.

9. The medical instrument according to claim 8, wherein the power drive is configured as a setting screw.

10. The medical instrument according to claim 1, wherein on the gripping surface of at least one jaw member that serves to receive a surgical needle that is to be held, a replaceable track of a synthetic material is positioned running in the longitudinal direction of the jaw member.

11. The medical instrument according to claim 1, wherein a replaceable covering made of a synthetic material can be placed on the gripping surface of at least one jaw member that serves to receive a surgical needle that is to be held.

12. The medical instrument according to claim 1, wherein at least the gripping surface of at least one jaw member that serves to receive a surgical needle that is to be held consists of a replaceable synthetic material.

13. The medical instrument according to claim 1, wherein the gripping members of the handle can be fixed in their respective position with respect to one another.
